(19) United States
(12) Patent Application Publication
(10) Pub. No.: US 2004/0029395 A1
(43) Pub. Date: Feb. 12, 2004

(54) Process solutions containing acetylenic diol surfactants

(76) Inventors: Peng Zhang, Quakertown, PA (US); Danielle Megan King, Emmaus, PA (US); Eugene Joseph Karwacki, Orefield, PA (US); Leslie Cox Barber, Cave Creek, AZ (US)

Correspondence Address: AIR PRODUCTS AND CHEMICALS, INC., PATENT DEPARTMENT, 7201 HAMILTON BOULEVARD, ALLENTOWN, PA, 181951501

(21) Appl. No.: 10/218,087

(22) Filed: Aug. 12, 2002

(51) Int. Cl. H01L 21/302; C07C 43/11; C07C 43/18; C07C 43/20

(52) U.S. Cl. 438/748; 568/616

(57) ABSTRACT

Process solutions comprising one or more acetylenic diol type surfactants are used to reduce the number of defects in the manufacture of semiconductor devices. In certain preferred embodiments, the process solution of the present invention may reduce post-development defects by improving the wetting of the solution on the surface of the patterned photoresist layer while minimizing foaming and bubble generation.

### BACKGROUND OF THE INVENTION

The present invention relates generally to methods for the manufacture of semiconductor devices. More specifically, the present invention relates to a method for reducing defects in semiconductor devices incurred during the manufacturing process without sacrificing throughput.

Defects are a major limiting factor for production yield and device function, particularly when the device sizes are reduced and wafer sizes are enlarged to 300 mm. The term “defects”, as used herein, relates to defects that may reduce the yield, or cause the loss, of the semiconductor device such as the collapse of the photoresist pattern on the substrate surface; particulates introduced onto the substrate resulting from processing such as lithography, etching, stripping, and chemical mechanical planarization (CMP) residues; particulates either indigenous to or resulting from manufacturing processes; pattern imperfections such as closed or partially open or blocked contacts or vias; line width variations; and defects resulting from poor adhesion of the resist to the substrate surface.

Pattern collapse is becoming an emerging problem in the production of semiconductor devices due to the higher aspect ratios in the new generation of devices. The thickness and aspect ratio of the patterned photoresist layer are important parameters for subsequent etch steps after lithography. At the 130 nm node, the aspect ratio for a photoresist layer having a 500 nm thickness may reach the value of 4. This value may be the point where the capillary force of the developer and/or rinse solution may lead to the collapse of the patterned photoresist layer. Besides capillary forces, the pattern collapse problem may be further influenced by other factors such as the mechanical strength of the resist, application of other coatings, i.e., anti-reflective coatings (ARC), and the nozzle type, position, and centrifugal forces during spin-on application of the photoresist layer.

The drive to reduce defects—thereby improving yield—presents new challenges to the manufacturing steps within the production of the semiconductor device, namely, the lithography, etching, stripping, and chemical-mechanical planarization (CMP) processes. The lithography process generally involves coating a substrate with a positive or negative photoresist, exposing the substrate to a radiation source to provide an image, and developing the substrate to form a patterned photoresist layer on the substrate. This patterned layer acts as a mask for subsequent substrate patterning processes such as etching, doping, and/or coating with metals, other semiconductor materials, or insulating materials. The etching process generally involves removing the surface of the substrate that is not protected by the patterned photoresist using a chemical or plasma etchant thereby exposing the underlying surface for further processing. The stripping process generally involves removing the cross-linked, photoresist pattern from the substrate via wet stripping or oxygen plasma ashing. The CMP process generally involves polishing the surface of the substrate to maintain flatness during processing. All of the aforementioned processes typically employ a rinse step to remove any particulate material that is generated from, or is a by-product of, these processes.

Reducing or eliminating the surface tension of the rinse liquid after pattern development may be used to reduce the capillary force that is exerted on the patterned photoresist layer. Two common approaches, to reduce or eliminate the surface tension of the rinse liquid, may be to freeze-dry the patterned photoresist features or employ supercritical fluids to dry the patterned photoresist layer after development. Both of these approaches may require extra manufacturing steps and special equipment that are not commonly used in semiconductor device fabrication.

A more common approach to reduce the surface tension may be to add a surfactant to the rinse liquid. The ability to reduce the surface tension of water at the air and liquid interface is of great importance in a variety of applications because decreased surface tension generally relates to increased wetting of water on the substrate surface. Surface tension reduction in water-based systems is generally achieved through the addition of surfactants. Equilibrium surface tension performance is important when the system is at rest, though the ability to reduce surface tension under dynamic conditions is of great importance in applications where high surface creation rates are used, i.e., spin coating, rolling, spray coating, and the like. Dynamic surface tension provides a measure of the ability of the solution to lower surface tension and provide wetting under high speed application conditions. Further, in certain applications such as during spray application, it is advantageous that the surfactant reduces the surface tension of the formulation in a manner that minimizes the problem of bubble generation and foaming. Foaming and bubble generation may lead to defects Consequently, considerable efforts have been made in the semiconductor industry towards solving the foaming problem.

Japanese patent JP 95142349A describes adding a fluorine-based surfactant such as ammonium perfluoroalkylsulfonate or perfluoroalkyl ethoxylate to the developer solution or rinse liquid.

U.S. Pat. No. 6,152,148 describes adding a surfactant such as a fluorosurfactant and a tetra alkyl quarternary ammonium hydroxide compound to an aqueous solution used to clean semiconductor wafers having a poly(arylene ether) dielectric film coating after CMP.

The article, Domke, W. D et al., “Pattern Collapse in High Aspect Ratio DUV- and 193 nm Resists”, Proc. SPIE-Int. Soc. Opt. Eng. 3999, 313-321, 2000 (“Domke”), describes adding surfactants to the developer solution to reduce the possibility of pattern collapse of acrylic and cycloolefin-maleic anhydride resists. The “surfactant” added to developer solution was the solvent, isopropyl alcohol. According to Domke, the addition of the “surfactant” in the developer solution did not have a consistent effect on pattern collapse.

PCT application WO 02/23598 describes adding the surfactant ammonium lauryl sulfate into the deionized (DI) water rinse and developer and applying them to a patterned photoresist to minimize or eliminate post-development defects.

Japanese Patent Application JP 96008163A describes adding hot water, an organic solvent, and a surfactant to a post-development rinse to prevent pattern collapse. No specific surfactants were mentioned.

PCT application 87/03387 describes protecting photoresist images against distortion or degradation by heat generated during etching and other processes by applying a thermally stabilizing, protective film to the substrate prior to the post-development bake of the image. Materials used for the film includes fluorocarbon surfactants, film forming polymers, chromium sulfate, trichloroacetic acid, chromotropic acid, and salts thereof.

The article, Cheung, C. et al. “A Study of a Single Closed Contact for 0.18 micron Photolithography Process” Proc. SPIE-Int. Soc. Opt. Eng. 3998, 738-741, 2000 (“Cheung”), discloses the use of surfactants such as octyl and nonyl phenol ethoxylates such as TRITON® X-114, X-102, X-45, and X-15, in the rinse solution to eliminate the photoresist residue and single closed contact defects. According to Cheung, the use of surfactant in the rinse solution did not provide much success.

U.S. Pat. No. 5,977,041 describes a post-stripping, aqueous rinse solution that includes water, a water soluble organic acid, and a water soluble surface-active agent. The surface-active agents include oligo(ethylene oxide) compounds having at least one aceylenic alcohol group.

WO 00/03306 describes a stripper composition that comprises an admixture of a solvent and a surfactant wherein the amount of solvent ranges from about 50 to about 99.9 weight percent of the total composition and the amount of surfactant ranges from about 0.1 to about 30 weight percent of the total composition.

Although surfactants have been commonly used as a post-development rinse solution, these solutions may not be effective in reducing the surface tension under dynamic conditions. Further, these solutions may have the undesirable side effect of foam generation. Because of these issues, the rinse solution using typical surfactants used in the art may not be effective in reducing all of the defects in the semiconductor device.

All references cited herein are incorporated herein by reference in their entirety.

### BRIEF SUMMARY OF THE INVENTION

The present invention satisfies some, if not all, of the needs of the art by providing a process solution and methods for using same. The surfactant within the process solution, present in a relatively minor amount, aids in removing particulates that may lead to defects through dispersion. In certain preferred embodiments, the process solution of the present invention may reduce post-development defects by improving the wetting of the solution on the surface of the patterned photoresist layer. The improved wetting of the process solution may aid in removing any particulates or residues left inside the contact holes or within dense features. Further, the process solution works more effectively in dynamic rinse situations with relatively minor foam generation compared to other surfactants presently used in the art.

Specifically, in one embodiment of the present invention, there is provided a method for reducing defects in the manufacture of semiconductor devices. The method comprises the steps of providing a substrate and contacting the substrate with a process solution comprising about 10 ppm to about 10,000 ppm of at least one surfactant having the formula (I) or (II):

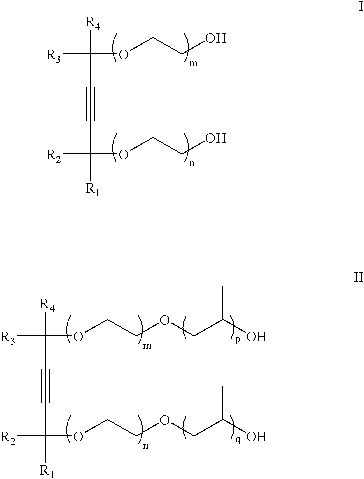

wherein R₁ and R₄ are a straight or a branched alkyl chain having from 3 to 10 carbon atoms; R₂ and R₃ are either H or an alkyl chain having from 1 to 5 carbon atoms; and m, n, p, and q are numbers that range from 0 to 20. In certain preferred embodiments, the process solution further comprises a dispersant.

In a further embodiment of the present invention, there is provided a method for reducing defects in the manufacture of semiconductor devices. The method comprises the steps of providing a substrate and contacting the substrate with a process solution comprising about 10 ppm to about 10,000 ppm of at least one surfactant having the formula:

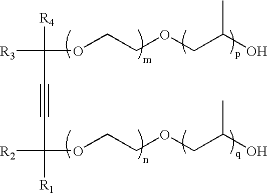

wherein R₁ and R₄ are a straight or a branched alkyl chain having from 3 to 10 carbon atoms; R₂ and R₃ are either H or an alkyl chain having from 1 to 5 carbon atoms; and m, n, p, and q are numbers that range from 0 to 20. In certain preferred embodiments, the sum of (p+q) of the surfactant ranges from 1 to 10.

In yet another embodiment of the present invention, there is provided a process solution having about 10 to about 10,000 ppm of at least one surfactant having the formula (I) or (II):

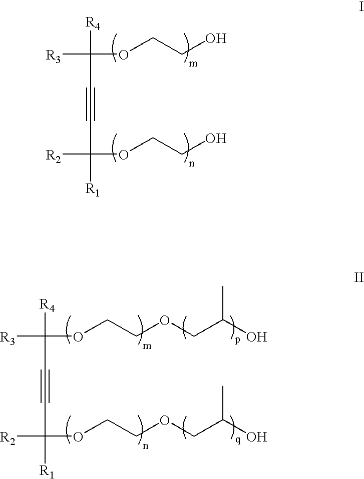

wherein R₁ and R₄ are a straight or a branched alkyl chain having from 3 to 10 carbon atoms; R₂ and R₃ are either H or an alkyl chain having from 1 to 5 carbon atoms; and m, n, p, and q are numbers that range from 0 to 20.

In a still further embodiment of the present invention, there is provided a process solution comprising about 10 to about 10,000 ppm of a surfactant having the formula:

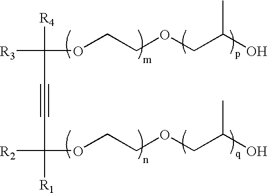

wherein R₁ and R₄ are a straight or a branched alkyl chain having from 3 to 10 carbon atoms; R₂ and R₃ are either H or an alkyl chain having from 1 to 5 carbon atoms; and m, n, p, and q are numbers that range from 0 to 20.

These and other aspects of the invention will become apparent from the following detailed description.

### DETAILED DESCRIPTION OF THE INVENTION

The present invention is directed to process solutions that are used to reduce the number of defects incurred during the manufacturing of the semiconductor device and methods of using same. The surfactant within the process solution aids in removing particulates that may lead to defects through dispersion. In certain preferred embodiments, the process solution of the present invention may reduce post-development defects by improving the wetting of the solution on the surface of the patterned photoresist layer. The improved wetting of the process solution may remove any residues left inside the contact holes or within dense features. Further, the process solution works more effectively in dynamic rinse situations with relatively minor foam generation compared to other surfactants presently used in the art.

The process solution of the present invention can be used in a variety of processes related to the manufacture of a semiconductor device such as for example, lithography process solutions, i.e., rinse, resist, edge bead remover, and anti-reflective coating (ARC) solutions; post-etching process solutions, i.e., sidewall film, stripper, post-strip/ash rinse solutions; CMP process solutions, i.e., slurry solution and post-CMP rinse solutions; wafer cleaning process solutions, i.e., additives to RCA or other standard cleaning solutions, super-critical CO₂ cleaning solutions, and solutions associated with ultra and megasonic cleaning; and process solutions for critical cleaning or precision cleaning for aerospace applications. The acetylenic diol derivatives surfactant within the process solution may allow for the reduction of equilibrium and dynamic surface tension while minimizing foaming.

The process solution of the present invention may be either aqueous-based or non-aqueous-based. The term “aqueous” as used herein, describes a solvent or liquid dispersing medium, which comprises at least 80 weight percent, preferably 90 weight percent, and more preferably at least 95 weight percent water. In embodiments wherein the process solution is aqueous-based, it is desirable that the acetylenic diol derived surfactant demonstrates a dynamic surface tension of less than 45 dynes/cm at a concentration of less than or equal to 5 weight percent in water at 23° C. and 1 bubble/second according to the maximum-bubble-pressure method of measuring surface tension described in Langmuir 1986, 2, 428-432, which is incorporated herein by reference in its entirety.

In embodiments where a solvent is used in addition to or in place of water, the solvent selected will not react with the acetylenic diol derived surfactant contained therein or the substrate. Suitable solvents include, but are not limited to, hydrocarbons (e.g. pentane or hexane); halocarbons (e.g. Freon 113); ethers (e.g. ethylether (Et₂O), tetrahydrofuran (“THF”), ethylene glycol monomethyl ether, or 2-methoxyethyl ether (diglyme)); nitrites (e.g. CH₃CN); or aromatic compounds (e.g. benzotrifluoride). Still further exemplary solvents include lactates, pyruvates, and diols. These solvents include, but are not limited to, acetone, 1,4-dioxane, 1,3-dioxolane, ethyl acetate, cyclohexanone, acetone, 1-methyl-2-pyrodidianone (NMP), and methyl ethyl ketone. Other solvents, include dimethylformamide, dimethylacetamide, N-methyl pyrrolidone, ethylene carbonate, propylene carbonate, glycerol and derivatives, naphthalene and substituted versions, acetic acid anyhydride, propionic acid and propionic acid anhydride, dimethyl sulfone, benzophenone, diphenyl sulfone, phenol, m-cresol, dimethyl sulfoxide, diphenyl ether, terphenyl, and the like. Still further solvents include propylene glycol propyl ether (PGPE), 3-heptanol, 2-methyl-1-pentanol, 5-methyl-2-hexanol, 3-hexanol, 2-heptano, 2-hexanol, 2,3-dimethyl-3-pentanol, propylene glycol methyl ether acetate (PGMEA), ethylene glycol, isopropyl alcohol (IPA), n-butyl ether, propylene glycol n-butyl ether (PGBE), 1-butoxy-2-propanol, 2-methyl-3-pentanol, 2-methoxyethyl acetate, 2-butoxyethanol, 2-ethoxyethyl acetoacetate, 1-pentanol, and propylene glycol methyl ether. The solvents enumerated above may be used alone or in combination with two or more solvents.

The process solutions of the present invention contain one or more nonionic surfactants that are acetylenic diol derivatives. The surfactants of the present invention may be represented by the following formula I or formula II:

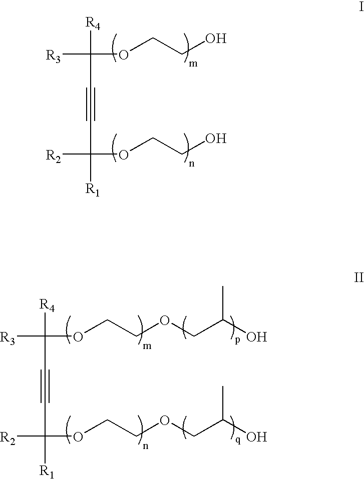

wherein R₁ and R₄ are a straight or a branched alkyl chain having from 3 to 10 carbon atoms; R₂ and R₃ are either H or an alkyl chain having from 1 to 5 carbon atoms; and m, n, p, and q are numbers that range from 0 to 20. The surfactants are commercially available from Air Products and Chemicals, Inc. of Allentown, Pa., the assignee of the present invention, under the trade names SURFYNOL® and DYNOL®. In certain preferred embodiments, the acetylenic diol portion of the molecule of formulas I or II is 2,4,5,9-tetramethyl-5-decyne-4,7-diolor2,5,8,11-tetramethyl-6-dodecyne-5,8-diol. The acetylenic diol derived surfactants of the present invention may be prepared in a number of ways including the methods described, for example, in U.S. Pat. No. 6,313,182 and EP 111 5035A1 which are assigned to the assignee of the present invention and incorporated herein by reference in their entirety.

In formula I and II, the alkylene oxide moieties represented by (OC₂H₄) are the (n+m) polymerized ethylene oxide (EO) molar units and the moieties represented by (OC₃H₆) are the (p+q) polymerized propylene oxide (PO) molar units. The value of (n+m) may range from 0 to 30, preferably from 1.3 to 15, and more preferably from 1.3 to 10. The value of (p+q) may range from 0 to 30, preferably from 1 to 10, and more preferably from 1 to 2.

In certain embodiments, the process solution may contain a dispersant. The amount of dispersant that is added to the process solution ranges from about 10 to about 10,000 ppm, preferably about 10 to about 5,000 ppm, and more preferably from about 10 to about 1,000 ppm. The term dispersant, as used herein, describes compounds that enhance the dispersion of particles such as dust, processing residue, hydrocarbons, metal oxides, pigment or other contaminants within the process solution. Dispersants suitable for the present invention preferably have a number average molecular weight that ranges from about 10 to about 10,000.

In certain preferred embodiments, the dispersant may be an ionic or a nonionic compound. The ionic or nonionic compound may further comprise a copolymer, an oligomer, or a surfactant, alone or in combination. The term copolymer, as used herein, relates to a polymer compound consisting of more than one polymeric compound such as block, star, or grafted copolymers. Examples of a nonionic copolymer dispersant include polymeric compounds such as the tri-block EO-PO-EO co-polymers PLURONIC® L121, L123, L31, L81, L101 and P123 (BASF, Inc.). The term oligomer, as used herein, relates to a polymer compound consisting of only a few monomer units. Examples of ionic oligomer dispersants include SMA® 1440 and 2625 oligomers (Elf Alfochem).

The dispersant may further comprise a surfactant. Typical surfactants exhibit an amphiphilic nature, meaning that they can be both hydrophilic and hydrophobic at the same time. Amphiphillic surfactants possess a hydrophilic head group or groups, which have a strong affinity for water and a long hydrophobic tail, which is organophilic and repels water. The surfactants may be ionic (i.e., anionic, cationic) or nonionic. Further examples of surfactants include silicone surfactants, poly(alkylene oxide) surfactants, and fluorochemical surfactants. Suitable non-ionic surfactants for use in the process solution include, but are not limited to, octyl and nonyl phenol ethoxylates such as TRITON® X-114, X-102, X-45, X-15 and alcohol ethoxylates such as BRIJ® 56 (C₁₆H₃₃(OCH₂CH₂)₁₀0H) (ICI), BRIJ® 58 (C₁₆H₃₃(OCH₂CH₂)₂₀OH) (ICI). Still further exemplary surfactants include alcohol (primary and secondary) ethoxylates, amine ethoxylates, glucosides, glucamides, polyethylene glycols, poly(ethylene glycol-co-propylene glycol), or other surfactants provided in the reference McCutcheon's Emulsifiers and Detergents, North American Edition for the Year 2000 published by Manufacturers Confectioners Publishing Co. of Glen Rock, N.J.

Various other additives may be optionally added to the process solution depending upon the application. These additives may include, but are not limited to, colorants, wetting agents, antifoamers, buffering agents, and other surfactants. Generally, unless otherwise stated, the amount of each of these additives would be about 0.0001 to 1 percent by weight, more preferably 0.0001 to 0.1 percent by weight, based upon the total weight of the process solution. In embodiments where one or more additional surfactants are added to the process solution, the surfactant may be any of the surfactants disclosed herein.

In certain embodiments, the process solution of the present invention may be used as a non-aqueous photoresist. In this connection, the process solution preferably comprises from 60 to 90, preferably from 70 to 90 weight percent solvent; from 5 to 40 weight percent, preferably from 10 to 20 weight percent resist polymer; from 0.5 to about 2 weight percent of a photoactive compound; 10 to 10,000 ppm of at least one acetylenic diol surfactant; and less than 1 weight percent of other additives such as polymerization inhibitors, dyes, plasticizers, viscosity control agents, and the like. The viscosity of the photoresist can be adjusted by varying the polymer to solvent ratio, thus allowing resists to be formulated for coating a variety of film thickness. Examples of suitable solvents within the photoresist process solution include any of the solvents contained herein. Non-limiting examples of a resist polymer include novolac resin or polyvinyl phenol copolymer. Non-limiting examples of a photoactive compounds include diazonaphthoquinone or photo acid generators (PAG).

The process solution of the present invention may also be used as a non-aqueous edge bead remover. Edge bead removers may be applied prior to baking the patterned photoresist layer to cross-link the polymer therein or prior to lithography. In this embodiment, the process solution preferably comprises from 99 to 100 weight percent solvent; 10 to 10,000 ppm of at least one acetylenic diol surfactant; and less than 1 weight percent of other additives. Examples of suitable solvents within the edge bead remover process solution include any of the solvents contained herein. In certain preferred embodiments, the solvent may be PGMEA, ethyl lactate, or anisole.

The process solution of the present invention may also be used as an anti-reflective coating for the top or bottom surface of the substrate. In this embodiment, the process solution preferably comprises from 60 to 99 weight percent solvent; from 1 to 40 weight percent, preferably 1 to 20 weight percent of a polymer; from 10 to 10,000 ppm of at least one acetylenic diol surfactant; and less than 1 weight percent of other additives such as crosslinker(s), surfactant(s), dye compounds, and the like. In general, the solids content of the process solution may vary from about 0.5 to about 40, preferably 0.5 to about 20, and more preferably 2 to 10 weight percent of the total weight of the process solution. Examples of suitable solvents within the ARC process solution include any of the solvents contained herein. In certain preferred embodiments, the solvent may be PGMEA or ethyl lactate. Examples of suitable polymers within the ARC process solution include, but are not limited to, acrylate polymers or phenyl-containing polymers such as those disclosed in U.S. Pat. No. 6,410,209 and spin-on-glass materials such as the methylsiloxane, methylsilsesquioxane, and silicate polymers such as those disclosed in U.S. Pat. Nos. 6,268,457 and 6,365,765.

The process solution of the present invention may be used in wafer cleaning methods, such as RCA-type cleaning, performed after the development step. In this embodiment, the substrate may be treated with the process solution after the stripping, CMP, ash cleaning, and/or etching steps have been completed. In one embodiment of the present invention, the process solution comprises a base such as an amine and/or ammonium hydroxide, alkylammonium hydroxide; an oxidizing agent such as H₂O₂; optionally a chelating agent; from 10 to 10,000 ppm of at least one acetylenic diol surfactant; and water. Some non-limiting examples of chelating agents are the following organic acids and its isomers and salts: (ethylenedinitrilo)tetraacetic acid (EDTA), butylenediaminetetraacetic acid, cyclohexane-1,2-diaminetetraacetic acid (CyDTA), diethylenetriaminepentaacetic acid (DETPA), ethylenediaminetetrapropionic acid, ethylenediaminetetrapropionic acid, (hydroxyethyl)ethylenediaminetriacetic acid (HEDTA), N, N, N′, N′-ethylenediaminetetra(methylenephosphonic) acid (EDTMP), citric acid, tartaric acid, phtalic acid, gluconic acid, saccharic acid, cathechol, gallic acid, pyrogallol, propyl gallate, and cysteine. In an alternative embodiment, the process solution comprises dilute HF; from 10 to 10,000 ppm of at least one acetylenic diol surfactant; and water. In a further embodiment, the process solution comprises an acid such as sulfuric acid or HCl and an oxidizing agent such as H₂O₂ wherein the ratio of the acid to the oxidizing agent is 1:1; optionally a chelating agent; from 10 to 10,000 ppm of at least one acetylenic diol surfactant; and water. In another embodiment, the process solution comprises electrolytic ionized water and from 10 to 10,000 ppm of at least one acetylenic diol surfactant. In yet another embodiment, the process solution comprises UV/ozone; from 10 to 10,000 ppm of at least one acetylenic diol surfactant; and water. For wafer cleaning applications, the process solution may be used for either megasonic or regular cleaning such as spray application.

The process solution of the present invention may be prepared by mixing the acetylenic diol derivative surfactant with water and/or other solvents and any additional additives. In certain embodiments, the mixing may be done at a temperature range of about 40 to 60° C. to affect dissolution of the ingredients contained therein. The resulting process solution may optionally be filtered to remove any undissolved particles that could potentially harm the substrate.

The process solution is preferably used to treat the surface of a substrate during or after the development step. Suitable substrates include, but are not limited to, materials such as gallium arsenide (“GaAs”), silicon, tantalum, copper, ceramics, aluminum/copper alloys, polyimides, and compositions containing silicon such as crystalline silicon, polysilicon, amorphous silicon, epitaxial silicon, silicon dioxide (“SiO₂”), silicon nitride, doped silicon dioxide, and the like. Further exemplary substrates include silicon, aluminum, or polymeric resins.

In certain preferred embodiments, the process solution is applied to a substrate having a photoresist coating applied thereto. The photoresist-coated substrate is then exposed to radiation to provide a design pattern that is imposed upon the photoresist coating. Depending upon whether the photoresist coating is positive or negative, the radiation either increases or decreased its solubility in a subsequently applied, alkaline developer solution such as a process solution containing tetramethylammonium hydroxide (TMAH). In a positive photoresist coating, the areas masked from radiation remain after development while the exposed areas are dissolved away. In a negative photoresist coating, the opposite occurs. The process solutions of the present invention may be suitable to treat substrates having either positive or negative photoresist coatings. After the patterned photoresist image is formed, the substrate is baked to harden the polymer contained within the photoresist.

The process solution is preferably applied to the surface of the substrate as a prepared solution. In alternative embodiments, however, the process solution can be prepared within the rinse stream just prior to or during contact with the substrate surface. For example, a certain quantity of one or more acetylenic diol derived surfactants can be injected into a continuous stream of water or other solvent medium that optionally includes other additives thereby forming the process solution. In some embodiments of the present invention, a portion of the acetylenic diol derived surfactant may be added to the substrate after application of the process solution. In this case, the process solution may be formed in multiple steps during the processing of the substrate. In still other embodiments of the present invention, the at least one surfactant can be also deposited upon or comprise the material of a high surface area device such as a cartridge or filter (which may or may not include other additives). A stream or water and/or solvent then pass through the cartridge or filter thereby forming the process solution. In still another embodiment of the present invention, the process solution is prepared during the contacting step. In this connection, at least one surfactant is introduced via a dropper or other means to the surface of the substrate. Water and/or other solvent medium is then introduced to the surface of the substrate and mixes with the at least one surfactant on the surface of the substrate thereby forming the process solution.

In an alternative embodiment of the invention, a concentrated composition is provided that may be diluted in water and/or other solvents to provide the process solution. A concentrated composition of the invention, or “concentrate” allows one to dilute the concentrate to the desired strength and pH. A concentrate also permits longer shelf life and easier shipping and storage of the product.

A variety of means can be employed in contacting the process solution with the substrate surface. The actual conditions of the contacting step (i.e., temperature, time, and the like) may vary over wide ranges and are generally dependent on a variety of factors such as, but not limited to, the nature and amount of residue on the surface of the substrate and the hydrophobicity or hydrophilicity of the substrate surface, etc. The contact step can be conducted in either a dynamic method such as, for example, a streamline process for applying the process solution over the surface of the substrate or in a static method such as, for example, a puddle rinse or immersing the substrate within a bath containing the process solution. The process solution may also be sprayed onto the surface of the substrate in a dynamic method such as in a continuous process or sprayed onto the surface and allowed to remain there in a static method. In certain preferred embodiments, the contacting step is conducted in a dynamic method. The duration of the conducting step, or time of contact of the process solution to the substrate surface, can vary from a fraction of a second to hundreds of seconds. Preferably, the duration can range from 1 to 200 seconds, preferably from 1 to 150 seconds, and more preferably from 1 to 40 seconds. The temperature range for the contacting step can vary from 10 to 100° C. and more preferably from 10 to 40° C.

The invention will be illustrated in more detail with reference to the following examples, but it should be understood that the present invention is not deemed to be limited thereto.

### EXAMPLES

### Examples 1 through 5

### Dynamic Surface Tension (DST)

Five process solutions containing acetylenic diol surfactants derived from 2,4,7,9-tetramethyl-5-decyne-4,7-diol (examples 1 through 3) or 2,5,8,11-tetramethyl-6-dodecyne-5,8-diol (examples 4 and 5) were prepared by adding 0.1 weight percent of the surfactant to deionized water under continuous stirring. The surfactants used in examples 1 through 5 are marketed by Air Products and Chemicals, Inc. of Allentown Pa., the assignee of the present invention, as SURFYNOL® 2502, SURFYNOL® 450, SURFYNOL® 104, DYNOL® 124, and DYNOL® 604, respectively.

The dynamic surface tension (DST) data for each process solution was collected via the maximum bubble pressure method described in Langmuir 1986, 2, pp. 428-432. The data was collected at bubble rates that range from 0.1 bubbles/second (b/s) to 20 b/s using the Kruss BP2 bubble pressure tensiometer manufactured by Kruss, Inc. of Charlotte, N.C. The molar units of EO and PO for each example and dynamic surface tension data is provided in Table I.

The dynamic surface tension data provides information about the performance of a surfactant at conditions from near-equilibrium (0.1 b/s) to relatively high surface creation rates (20 b/s). For applications such as semiconductor or IC processing, high bubble rates may correspond to a faster substrate rotation speed or a dynamic dispense in a post-development rinse process. It is desirable that the dynamic surface tension by reduced below that of water at high bubble rates (i.e., 70-72 dyne/cm at 20 b/s) to provide, inter alia, better wetting of the photoresist-coated substrate, reduction in the number of defects, and prevention of pattern collapse. As Table I illustrates, all of the process solutions exhibited dynamic surface tensions at high bubble rates below that of water. This indicates that the process solutions of the present invention may be effective at reducing the surface tension of water.

TABLE I
Dynamic Surface Tension

| | | | DST | DST | DST | DST | DST |
|---|---|---|---|---|---|---|---|
| Ex- | Moles | Moles | (dyne/ | (dyne/ | (dyne/ | (dyne/ | (dyne/ |
| am- | EO | PO | cm) | cm) | cm) | cm) | cm) |
| ple | (m + n) | (p + q) | 0.1 b/s | 1 b/s | 6 b/s | 15 b/s | 20 b/s |
| 1 | 5 | 2 | 34.0 | 35.3 | 37.6 | 41.5 | 44.3 |
| 2 | 5 | 0 | 35.1 | 35.2 | 38.1 | 42.0 | 44.4 |
| 3 | 0 | 0 | 32.1 | 33.1 | 34.2 | 36.1 | 40.3 |
| 4 | 0 | 0 | 34.1 | 43.6 | 58.1 | 68.3 | 69.8 |
| 5 | 4 | 0 | 26.8 | 26.8 | 31.5 | 35.9 | 39.1 |

### Examples 5 through 7

### Foaming Properties

Three process solutions containing acetylenic diol surfactants derived from 2,4,7,9-tetramethyl-5-decyne-4,7-diol (examples 5 and 6) or 2,5,8,11-tetramethyl-6-dodecyne-5,8-diol (example 7) were prepared by adding 0.1 weight percent of each surfactant to deionized water under continuous stirring. The surfactants used in examples 5 through 7 are marketed by Air Products and Chemicals, Inc. of Allentown Pa., the assignee of the present invention, as SURFYNOL® 2502, SURFYNOL® 104, DYNOL® 604, respectively.

Foaming is an undesirable side effect of surfactants in rinse solution. The foaming properties of examples 5 through 7 were examined using a procedure based upon ASTM D 1173-53, the Ross-Miles test method, and the results are provided in Table II. In this test, a 200 ml quantity of each process solution is added from an elevated foam pipette to a foam receiver containing the 50 ml of the same solution at room temperature. The Ross-Miles method stimulates the action of pouring a liquid into a cylindrical vessel containing the same liquid. The results are given in Table II. The foam height is measured at the completion of the addition (“Initial Foam Height”) and the time required for the foam to dissipate is recorded (“Time to 0 Foam”). In certain applications, foam may be undesirable because it may lead to defects due to the failure to adequately coat the surface of the substrate. As Table II indicates, the time to reach zero foam is approximately one minute or less.

The process solution of Example 5 was also compared to process solutions containing 0.1 weight percent of a fluorosurfactant (perfluoroalkyl ethoxylate) and an ionic surfactant (sodium lauryl sulfate) using the Ross-Miles test. The results of this comparison are provided in Table III. As Table III shows, solutions containing the fluorosurfactant and ionic surfactant still exhibited significant foam at intervals of 5 or 10 minutes. In semiconductor processing applications, the presence of significant foam may be undesirable and may lead to an increase in processing defects.

TABLE II
Foaming Properties

| | Moles EO | Moles PO | Initial Foam | Time to Zero |
|---|---|---|---|---|
| Example | (m + n) | (p + q) | Height (cm) | Foam (sec) |
| 5 | 5 | 2 | 0.6 | 6 |
| 6 | 0 | 0 | 2.0 | 3 |
| 7 | 4 | 0 | 2.5 | 60 |

TABLE III
Comparison of Foam Properties with Solutions containing other
Surfactants

| | | Foam | Foam | Foam |
|---|---|---|---|---|
| Rinse | Initial Foam | Height at | Height at | Height at |
| Composition | Height (cm) | 6 sec (cm) | 5 min (cm) | 5 min (cm) |
| Example 5 | 0.6 | 0 | 0 | 0 |
| Fluorosurfactant | 14.5 | 14.5 | N/A | 13.5 |
| (0.1 weight %)⁽¹⁾ | | | | |
| Ionic surfactant | 22.0 | 22.0 | 20.0 | N/A |
| (0.25 weight | | | | |
| %)⁽²⁾ | | | | |

⁽¹⁾Information obtained from DuPont ZONYL ® marketing literature.
⁽²⁾Information obtained from Weil, J. K., et al., “Synthetic Detergents from Animal Fats: the Sulfonation of Tallow Alcohols”, J. Am. Oil Chem. Soc. 31, p. 444-47 (1954).

### Examples 8 through 9

### Contact Angle Data

The wettability of process solutions containing varying amounts of surfactants derived from 2,4,7,9-tetramethyl-5-decyne-4,7-diol (examples 8a and 8b) or 2,5,8,11-tetramethyl-6-dodecyne-5,8-diol (examples 9a and 9b) and DI water as a comparison (comparative example 1) was measured on the G10/DSA10 Kruss drop shape analyzer provided by Kruss USA of Charlotte, N.C. using the Sessile drop method. In this method, the wetting properties of a localized region on the surface of a photoresist-coated substrate are estimated by measuring the contact angle between the baseline of a droplet of aqueous developer solution and the tangent at the droplet base. A high-speed camera captured the spreading of the droplet at a speed of 2 frames per second for 2 minutes and the contact angle was measured.

Process solutions of surfactant based on 2,4,7,9-tetramethyl-5-decyne-4,7-diol and 2,5,8,11-tetramethyl-6-dodecyne-5,8-diol, or SURFYNOL® 2502 and DYNOL® 604 provided by Air Products and Chemicals, Inc. of Allentown, Pa., were prepared in the following manner. A volumetric flask was charged with varying amounts of the surfactant and DI water to reach a level of 100 ml at room temperature. The mixture was agitated until the surfactant was dissolved therein to form the process solution. The amounts of surfactant in the process solutions of examples 8a, 8b, 9a and 9b are provided in Table IV.

Silicon wafers provided by Wafernet Inc. of San Jose, Calif. were coated with a AX 4318 photoresist coating provided by Sumitomo Chemical Co., Ltd. of Osaka, Japan using a spin coating process at a spin speed of 3200 rpm. The contact angle of the process solution on the photoresist surface was measured. Table IV provides the value of the contact angle for the process solutions and DI water (comparative example 1) at different drop ages expressed in seconds.

In general, contact angles of about 20° or below may indicate complete wetting of the substrate surface. As Table IV illustrates, the contact angles of TMAH developer on the photoresist-coated substrate that were treated with the process solutions of the present invention are smaller than the contact angle of the photoresist treated with DI water. Further, higher amounts of surfactant within the process solution may lead to more surfactant adsorption and improved wetting.

TABLE IV

| | | Contact | Contact | Contact | Contact |
|---|---|---|---|---|---|
| | Amt | Angle | Angle | Angle | Angle |
| Example | Surfactant | (0 sec) | (5 sec) | (10 sec) | (30 sec) |
| Comp. Ex. 1 - DI | — | 61.8 | 61.7 | 61.5 | 61.1 |
| water | | | | | |
| Ex. 8a | 125 ppm | 47.3 | 46.9 | 46.5 | 45.4 |
| Ex. 8b | 600 ppm | 47.3 | 42.6 | 40.6 | 36.4 |
| Ex. 9a | 100 ppm | 50.0 | 46.8 | 45.0 | 41.6 |
| Ex. 9b | 350 ppm | 40.0 | 29.4 | 25.3 | 17.2 |

### Example 10

### Number of Post-Development Defects after DI Rinse vs. Process Solution Rinse

The number of post-development defects on a substrate was compared after treating the substrate with a rinse of DI water (comparative example 2) vs. a rinse containing the process solution of the present invention (example 10). The process solution contained 50 ppm of a 2,5,8,11-tetramethyl-6-dodecyne-5,8-diol-derived surfactant, or DYNOL® 604 provided by Air Products and Chemicals, Inc. of Allentown, Pa., and 170 ppm of the oligomer dispersant SMA® 1440 provided by Elf Alfochem. The substrate was processed in the following manner: a photoresist-coated substrate was exposed to a 365 nm light, heated to a temperature of approximately 110° C. for a time of about 1 minute and then developed to form a patterned photoresist with a dilute TMAH solution. The TMAH solution was applied by dynamically dispensing a 0.21N TMAH solution onto the substrate for a period of 100 seconds.

In comparative example 2, a rinse containing DI water started 15 seconds before the developer nozzle was turned off and continued for a period of 7 minutes. The substrate was inspected for defects using the TereStar® KLA-Tencor defect inspection tool provided by KLA-Tencor Inc. of San Jose, Calif. and the defects were classified and counted. The results of the inspection are provided in Table V.

The substrate was processed in the same manner as in comparative example 2 using the same developer and process conditions. However, after 100 seconds of developing, a process solution comprising an acetylenic diol surfactant (example 10) was used to rinse the patterned photoresist-coated surface. The overlapping period with the developer was the same as in comparative example 2. After a 120 second rinse with the process solution, a DI water rinse was used for another 7 minutes. The substrate was inspected for defects using the TereStar® KLA-Tencor defect inspection tool and the defects were classified and counted. The results of the inspection are provided in Table VI.

As Table VI illustrates, the process solution of the present invention was able to completely remove the photoresist residues from the patterned photoresist surface. By contrast, Table V shows that were many defects resulting from residual photoresist and other sources after rinsing with DI water. Therefore, rinsing the substrate with the process solution of the present invention effectively eliminated the number of post-development defects and improved the process yield.

TABLE V
Post-Development Defects after DI Water Rinse

| Defect Types | Small | Medium | Large | Extra large | Total |
|---|---|---|---|---|---|
| Pattern Defect | 0 | 55 | 35 | 1 | 91 |
| Pinholes/Dots | 0 | 148 | 2 | 0 | 150 |
| Total | 0 | 203 | 37 | 1 | 241 |

Table VI
Post-Development Defects after Process solution Rinse

| Defect Types | Small | Medium | Large | Extra large | Total |
|---|---|---|---|---|---|
| Pattern Defect | 0 | 0 | 0 | 0 | 0 |
| Pinholes/Dots | 0 | 0 | 0 | 0 | 0 |
| Total | 0 | 0 | 0 | 0 | 0 |

### Example 11

### Comparison of Equilibrium Surface Tension and Dynamic Surface Tension of Process solution vs. Solutions Containing Fluorosurfactant

Process solutions containing 0.1 weight percent of a surfactant derived from 2,5,8,11-tetramethyl-6-dodecyne-5,8-diol, or DYNOL® 604 provided by Air Products and Chemicals, Inc. of Allentown, Pa. (example 11), and a fluorosurfactant, potassium perfluorooctane carboxylate provided by 3M of St. Louis, Mo. were prepared in order to compare the equilibrium surface tension (EST) and dynamic surface tension (DST). The EST for both solutions was measured using the Wilhemy plate method on a Kruss BP2 bubble pressure tensiometer manufactured by Kruss, Inc. of Charlotte, N.C. The DST of each process solution was measured via the maximum bubble pressure method used in examples 1 through 5. The results of the EST and DST tests are provided in Table VII.

Referring to Table VII, while the fluorosurfactant exhibits a lower EST compared to the process solution of the present invention, the significantly lower DST indicates that the fluorosurfactant exhibits poor dynamic surface tension reduction ability. For applications that require high surface creation rates such as dynamic rinse processes used in semiconductor manufacturing, the process solution of the present invention would be more suitable than solutions containing fluorosurfactants due to its lower DST value.

TABLE VII

| Rinse Composition (0.1 wt %) | EST (dyne/cm) | DST (cm/cm) |
|---|---|---|
| Example 11 | 25.8 | 28.4 |
| Fluorosurfactant | 21.2 | 72.4 |

While the invention has been described in detail and with reference to specific examples thereof, it will be apparent to one skilled in the art that various changes and modifications can be made therein without departing from the spirit and scope thereof.

We claim:

1. A method for reducing defects during the manufacture of semiconductor devices, the method comprising:
providing a substrate; and
contacting the substrate with a process solution comprising about 10 ppm to about 10,000 ppm of at least one surfactant having the formula (I) or (II): 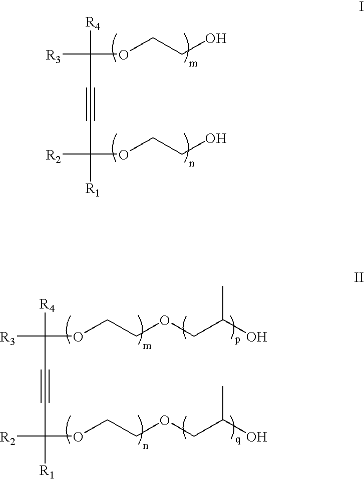
wherein R₁ and R₄ are a straight or a branched alkyl chain having from 3 to 10 carbon atoms; R₂ and R₃ are either H or an alkyl chain having from 1 to 5 carbon atoms; and m, n, p, and q are numbers that range from 0 to 20.

2. The method of claim 1 wherein the process solution further comprises from about 10 to about 10,000 ppm of at least one dispersant.

3. The method of claim 2 wherein the at least one dispersant comprises a nonionic compound.

4. The method of claim 2 wherein the at least one dispersant comprises an ionic compound.

5. The method of claim 4 wherein the at least one dispersant comprises a surfactant.

6. The method of claim 1 wherein the value of (n+m) ranges from 0 to 30.

7. The method of claim 6 wherein the value of (n+m) ranges from 1.3 to 15.

8. The method of claim 1 wherein the value of (p+q) ranges from 0 to 30.

9. The method of claim 6 wherein the value of (p+q) ranges from 1 to 10.

10. The method of claim 1 wherein the contact angle is about 600 or less at 30 seconds.

11. The method of claim 10 wherein the contact angle is about 500 or less at 30 seconds.

12. The method of claim 11 wherein the contact angle is about 400 or less at 30 seconds.

13. The method of claim 1 wherein the contacting step comprises a dynamic rinse.

14. The method of claim 13 wherein the process solution exhibits a dynamic surface tension of about 45 dynes/cm² or less at 23° C. and 1 bubble/second according to the maximum-bubble-pressure method.

15. The method of claim 13 wherein the process solution exhibits substantially zero foam at a time greater than 60 seconds.

16. A method for reducing defects during the manufacture of semiconductor devices, the method comprising:
providing a substrate; and
contacting the substrate with a process solution comprising about 10 ppm to about 10,000 ppm of at least one surfactant having the formula: 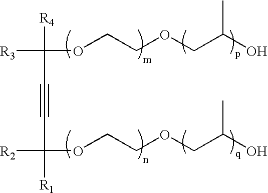
wherein R₁ and R₄ are a straight or a branched alkyl chain having from 3 to 10 carbon atoms; R₂ and R₃ are either H or an alkyl chain having from 1 to 5 carbon atoms; and m, n, p and q are numbers that range from 0 to 20.

17. A process solution, the solution comprising:
about 10 to about 10,000 ppm of at least one surfactant having the formula (I) or (II): 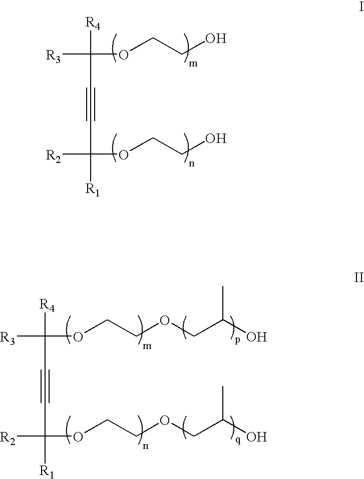
wherein R₁ and R₄ are a straight or a branched alkyl chain having from 3 to 10 carbon atoms; R₂ and R₃ are either H or an alkyl chain having from 1 to 5 carbon atoms; and m, n, p, and q are numbers that range from 0 to 20.

18. The process solution of claim 17 wherein the process solution further comprises from about 10 to about 10,000 ppm of at least one dispersant.

19. The process solution of claim 18 wherein the at least one dispersant comprises a nonionic compound.

20. The process solution of claim 18 wherein the at least one dispersant comprises an ionic compound.

21. The process solution of claim 17 wherein the value of (n+m) ranges from 0 to 30.

22. The process solution of claim 21 wherein the value of (n+m) ranges from 1.3 to 15.

23. The process solution of claim 17 wherein the value of (p+q) ranges from 0 to 30.

24. The process solution of claim 23 wherein the value of (p+q) ranges from 1 to 10.

25. The process solution of claim 17 further comprising a photoactive compound.

26. The process solution of claim 17 further comprising a solvent.

27. The process solution of claim 17 further comprising a polymer.

28. The process solution of claim 17 further comprising a base.

29. The process solution of claim 17 further comprising an acid.

30. A process solution, the solution comprising:
about 10 to about 10,000 ppm of at least one surfactant having the formula: 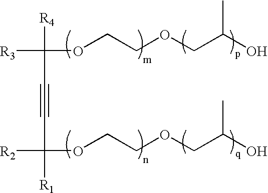
wherein R₁ and R₄ are a straight or a branched alkyl chain having from 3 to 10 carbon atoms; R₂ and R₃ are either H or an alkyl chain having from 1 to 5 carbon atoms; and m, n, p, and q are numbers that range from 0 to 20.
